(19) United States
(12) Patent Application Publication
(10) Pub. No.: US 2015/0182708 A1
(43) Pub. Date: Jul. 2, 2015

(54) Apparatus and Method for Mitigation of Smoke and Particulate in Minimally Invasive Surgery

(71) Applicant: Joshua C. Barnard, Las Vagas, NV (US)

(72) Inventor: Joshua C. Barnard, Las Vagas, NV (US)

(21) Appl. No.: 14/536,773

(22) Filed: Nov. 10, 2014

Related U.S. Application Data
(60) Provisional application No. 61/922,216, filed on Dec. 31, 2013.

(51) Int. Cl. A61M 13/00; B03C 3/017

(52) U.S. Cl. CPC A61M 13/003; B03C 3/017; A61M 2205/75; B03C 2201/26

(57) ABSTRACT

A device for the mitigation of smoke and particulate suspended in an insufflated body cavity during minimally invasive surgical procedures consist of a single electrode manufactured within and/or on a trocar shaft, or any other laparoscopic or endoscopic instrument. The electrode is made of any electrically active material with many hundreds or thousands of extremely fine terminal points, such as carbon fiber or laser etched metals. The electrode is electrically connectable to the neutral (negative) pole of a source of high voltage direct current (DC) electricity and the positive pole of the same electrical source is grounded or terminally insulated. When the positive electrical pole is connected to the ground and the carbon fiber or etched metal electrode is connected the negative pole, anions stream from the terminal ends of the terminal ends attracting smoke, and any other particulate suspended in the cavity, causing them to precipitate out of the suspending gas and therefore clearing the surgeon's view.

This is a regular utility patent application which claims priority to co-pending U.S. Provisional Patent Application Ser. No. 61/922,216, filed Dec. 31, 2013, and which is incorporated herein by reference for all purposes.

### FIELD OF THE INVENTION

This invention relates to the field of laparoscopic and other minimally invasive surgical procedures. The apparatus and method of the present invention clears and keeps clear the view of surgeons performing minimally invasive surgical procedures by forcing smoke, particulate and condensation out of suspension within a gas filled body cavity.

### BACKGROUND OF THE INVENTION

Smoke, for the purposes to this application is operationally defined as particles suspended in a gas, having been created by an electrocautery, electrosurgical or a laser device, which may obscure a surgeon's view of the surgical site.

Smoke generated during intracorporeal procedures has been a problematic issue since the inception of minimally invasive surgery. The peritoneum, or any other body cavity which surrounds the surgical site of interest, is inflated with an inert gas such as CO₂ so that a level of separation between viscera and cavity walls may be obtained. The separation is necessary for visibility and maneuverability of instrumentation. A pressurized tank not unlike a scuba or welding tank insufflates the body cavity via a pressure regulation valve.

There are two current methods for the removal of smoke from an insufflated body cavity. The first of these methods is simply to vent the gas from the peritoneal cavity. Venting gas is relatively ineffective at clearing the surgeon's view of the surgical site, and it adds a significant amount of time to a procedure. Also, venting gas causes the temporary loss of pneumoperitoneum. As the peritoneum deflates, the surgeons view changes due to the collapse of the cavity around the surgical site. This collapse lowers the intracorporeal pressure, which opens the pressure-regulating valve, and through fluid communication with the body cavity, begins to insufflate. The second currently employed method involves the vacuum withdrawal of gas. The vacuum systems are cumbersome, inconvenient and must be set up and utilized properly in order to avoid giving the patient a potentially fatal embolism. Both of the methods involve removing the gas from the peritoneum while replacing it with new gas. Aside from the less than desirable efficacy, these methods tend to dry out the body cavity and visceral tissue.

Time in an operating room cost a significant amount of money. When surgeons stop multiple times in the course of a procedure to vent gas, that time is added to the bill and detracts from the surgeon's ability to complete further procedures in the same day. Furthermore, when a surgeon is operating with poor visibility, this too adds time to the procedure and adds stress to the surgeon's workday. This phenomenon is similar to driving an automobile in a fog, one arrives at their destination more slowly and feeling more stress and fatigue than when driving on a clear day.

There are a number of patents and patent applications which relate to the problem of removing smoke from minimally invasive surgical sites. However, the overwhelming majority of these specifications disclose transfer of gas either through venting or vacuuming the smoke charged gas from the peritoneum and replacing the removed gas with clear gas. US Publication No. US-2012-0067212A1, published Mar. 22, 2012, specifies an electrical method of smoke reduction and removal.

### SUMMARY OF INVENTION

The apparatus of the present invention is comprised of or includes a single carbon fiber or etched metal surface electrode in communication with, or connectable to, the negative (neutral) pole of a source of high voltage direct current (DC) electricity. The positive pole of the same source of DC electricity is given the least resistive path to an isolated (unshared) ground. When the device is charged, anions (negative ions) stream from the terminal ends of carbon fibers or etched metal barbs into the insufflated body cavity and force suspended particles out of suspension. In this way, the invention clears and keeps clear the vision of an operating surgeon.

This invention is not limited to being embedded in a trocar, but the trocar design is a preferred embodiment. The working prototypes show the most versatility, convenience and efficacy in the trocar embodiment. However, there may be affirmative results with the present invention affixed to and embodied in several other laparoscopic and endoscopic devices.

The trocar-embodied device is preferable for various reasons. Importantly, the trocar is integral in laparoscopy and at least one trocar is employed in almost every laparoscopic procedure. Thus, the ability to clear smoke is available without regard the instrument being deployed through the trocar. Furthermore, the modern disposable trocar's polycarbonate construction allows for a level of insulation and disposability.

The carbon fiber or etched metal electrode of the present invention may be manufactured into the trocar. The trocar is used in its usual fashion, but once entry into the patient's body has been made and insufflation of the cavity has begun, the trocar and it's on-board carbon fiber or etched metal electrode can be connected via a small plug to the rest of the apparatus, completing the device. The most attractive prototype does not plug into anything external because the entire apparatus is manufactured inside of the trocar. in this embodiment, once entry into the peritoneum is made and insufflation has begun, a small switch on the trocar is moved to the “on” position. This is a great advantage in that it adds nothing to the surgical field. The prior art publication No. US-2012-00672A1 device has several parts which it adds to the surgical field and relies on bipolar “ionization” of particles as opposed to this inventive device's negative ion (anion) generation method. The present inventive device is intended to be prophylactic, when running constantly. However, it can be turned on in emergent fashion after smoke has completely obscured the surgeon's vision. The preference for running the device constantly is several-fold. The invention is intended to prevent the surgeon from having to stop operating. The surgeon should never need to avert his or her eyes from the surgical site. The intention of the present inventive device is to reduce steps in a surgical procedure, not to add steps, or trade one step for another.

While the above referenced prior art publication discloses the overarching idea of ionization of gas in a local atmosphere in order to address the issue of smoke in laparoscopy, there are several fundamental differences in design and theory between the prior art apparatus and the present inventive apparatus and method.

The present inventive apparatus and method utilizes a single electrode in communication with only the negative (neutral) pole of the high voltage source of DC electricity. The positive pole of the same source of high voltage DC electricity has no electrode and is put directly to a separate ground or terminally insulated isolation, which is not shared with any other device or the patient. The positive pole is intentionally given the least resistive route to an isolated ground in order to avoid the tendency toward arching and the rogue creation of free radicals, such as trioxide gas, within the patient. The prior art publication specifies the use of both the positive and negative poles and it appears, at least at times, the positive pole shares the patient's grounding pad, which may lead to the unwanted production of free radicals.

The carbon fiber electrode array or etched metal foil employed within the present inventive device may be comprised of hundreds or thousands of terminal projection points from which to stream anions. Having more than one point allows for a less resistive path into space for the anions. Thus more anions are produced for a given voltage. This discourages arching and reduces the rogue formation of trioxide (ozone) molecules. Also the increase in terminal points of these electrodes lowers the voltage and current required to produce the number of anions necessary for efficacy in clearing smoke and particulate in the pneumoperitoneum. This advance has allowed the inventor to miniaturize the entire apparatus and lower the manufacturing costs to the point that it can be completely encased in a disposable trocar.

The present inventive apparatus and method employs only the negative pole electrode with the positive pole connected to an isolated and unshared ground or is isolated internally in a terminal insulator. The device does not comprise a completed circuit and therefore does not create a significant current. Thus, the present device does not require a current monitoring or regulating component for patient safety. While the final designs may vary, the working prototypes have been powered effectively by small batteries producing an input voltage of 5 volts DC and an input current between 5 milliamps (mA) and 176 mA and an output current not exceeding 250 microamps (μA). The working prototype may be touched directly without arching or discomfort.

The present device disclosed herein utilizes only the negative pole of a source of high voltage DC electricity, and the positive pole of the same source of DC electricity is in no way connectable, in communication with, or even in the proximity of the negatively charged electrode or the patient. Therefore, the present invention may act as a non-directional anion generator. The inventive device produces an abundance of anions in the pneumoperitoneum. These anions react with the net positively charged smoke particles suspended in the pneumoperitoneum causing the particles to come together or clump together in an ionic bond. The increasing mass causes the suspended particle to come together to precipitate from the suspending gas. The prior publication specifies a bi-polar device, which streams electrons from one electrode to the other attracting “ionized” particles toward the positively charged electrode or the patient's positively charged main body.

The present inventive apparatus and method utilizes a single electrode. Therefore, it may be used in a single site surgical procedure.

The present trocar-embedded electrode may be use utilized with any laparoscopic surgical device without requiring each surgical device to employ the technology. Furthermore, it allows for the electrosurgical devices to be completely separate from this invention. Most, if not all, electrosurgical and electrocautery devices only allow for one operation at any given time. This is to avoid dangerous voltage and current spikes during operation. The trocar design keeps the electrical operation of all other surgical devices completely separate from the electrical operation of this invention.

### BRIEF DESCRIPTION OF THE DRAWINGS

FIG. 1 illustrates an overview of the apparatus and method in relation to a patient P and the electrical connection of the components.

FIG. 2 shows the DC power supply details.

FIG. 3 shows the AC power supply details.

FIG. 4 illustrates the trocar shaft and carbon fiber electrode details.

FIG. 5 is a drawing of the peritoneum with the apparatus inserted.

FIG. 6 depicts the latest prototype in which the entire apparatus is encompassed and integrated into the trocar.

FIG. 7 is schematic of the apparatus miniaturized to fit in a disposable trocar.

### GLOSSARY OF TERMS USED IN THE PRESENT DISCLOSURE

Abdominal Cavity—The largest body cavity in humans. The cavity is bound by the thoracic diaphragm (superior), and the pelvic floor (inferior), and it holds most of the viscera (stomach, liver, gallbladder, spleen, pancreas, small intestine, kidneys, large intestine, adrenal glands and some reproductive organs).

Anions—An ion with more electrons than protons, giving it a net negative charge.

AC/DC Step-up Transformer—An electrical device used to convert an alternating current input to a higher voltage direct current output.

Carbon Fiber Electrode—An electrical device comprised of many hundreds or thousands of hair like carbon fibers used to project anions (negative ions) into space.

DC Boost Converter—A electrical device with a direct current (DC) output voltage that is higher than the DC Input voltage.

Device—The invention disclosed herein.

Ground—A direct physical connection to Earth.

Electrical Filament—A thin, highly conductive wire carrying anions down the trocar shaft to the carbon fiber electrode.

Electrocautery—Also known as thermal cautery, is the process of heating living tissue to achieve homeostasis and/or varying degrees of tissue destruction. This process of burning tissue invariably produces significant smoke and steam.

Electrosurgery—A group of commonly used procedures that utilize the passage of very high frequency alternating electrical current through living tissue to achieve varying degrees of tissue destruction and achieve homeostasis during dissection. While electrosurgical devices produce less smoke than conventional thermal cautery, they do present the same problem.

Etched Metal Electrode—A metal surface which is etched, often by laser, to create many thousands of microscopic points from which to stream anions.

Interruption Switch—A switch which opens and closes an electrical circuit.

Insufflation—The process of introducing gas, such as CO2 into the abdominal cavity under regulated pressure in order to achieve pneumoperitoneum (see pneumoperitoneum).

Laparoscope—A slender endoscopic camera inserted through a trocar into the peritoneum for viewing the abdominal and pelvic cavities.

Peritoneum—The serous membrane that forms the lining of the abdominal cavity.

Pneumoperitoneum—Air or gas in the peritoneal cavity. For the purpose of this article the air or gas is intentionally introduced into the body to achieve the separation between the abdominal/peritoneal wall and the visceral tissues necessary to perform minimally invasive surgical procedures.

Trocar—A pointed instrument, fitted inside of a hollow tube or cannula, used to directly enter the abdominal cavity. The pointed instrument is then removed, leaving a hollow port of entry for surgical instruments used in minimally invasive surgical procedures. Note: Modern trocars are equipped with cannula and valves for use in the process of insufflation (see insufflation).

### DESCRIPTION OF A PREFERRED EMBODIMENT

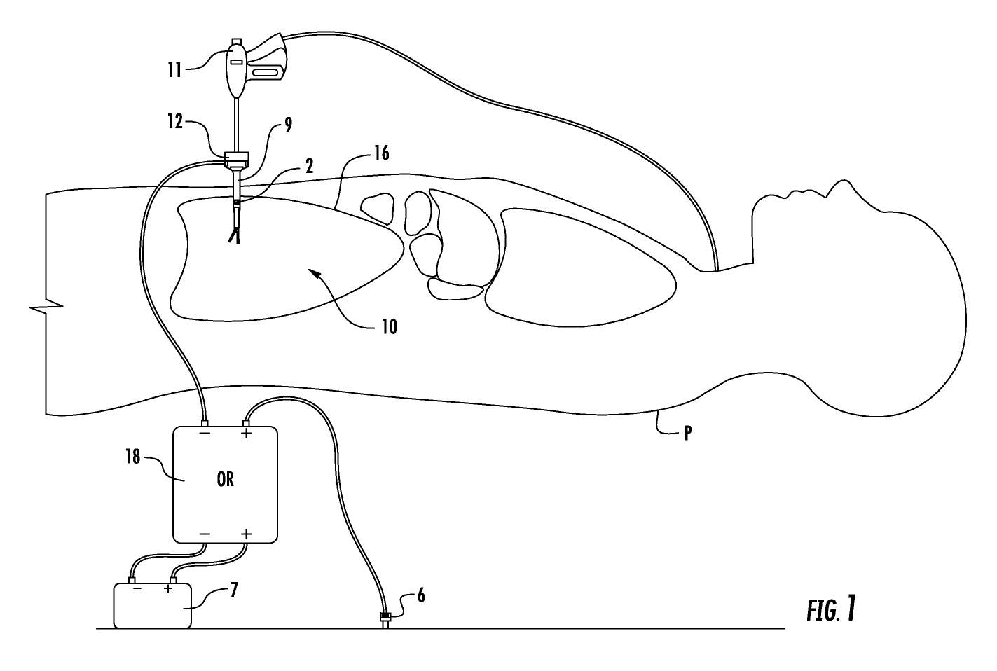

Turning to the drawings the present invention may be more fully understood. FIG. 1 illustrates that an energy source 7 supplies a high voltage source 18 with power. The high voltage source generates an output with a higher voltage than the input voltage. The now higher voltage is output as DC electricity at the positive + and negative − terminals. The positive + pole is permanently connected directly to an isolated and unshared ground 6. The negative − terminal is electrically connected to a carbon fiber electrode 2, which is affixed to or embedded in a trocar 12. The trocar shaft 9 is a hollow tube, which pierces the patient's P peritoneum 16 acting as a port of entry into the pneumoperitoneum for electrocautery/electrosurgical devices 11.

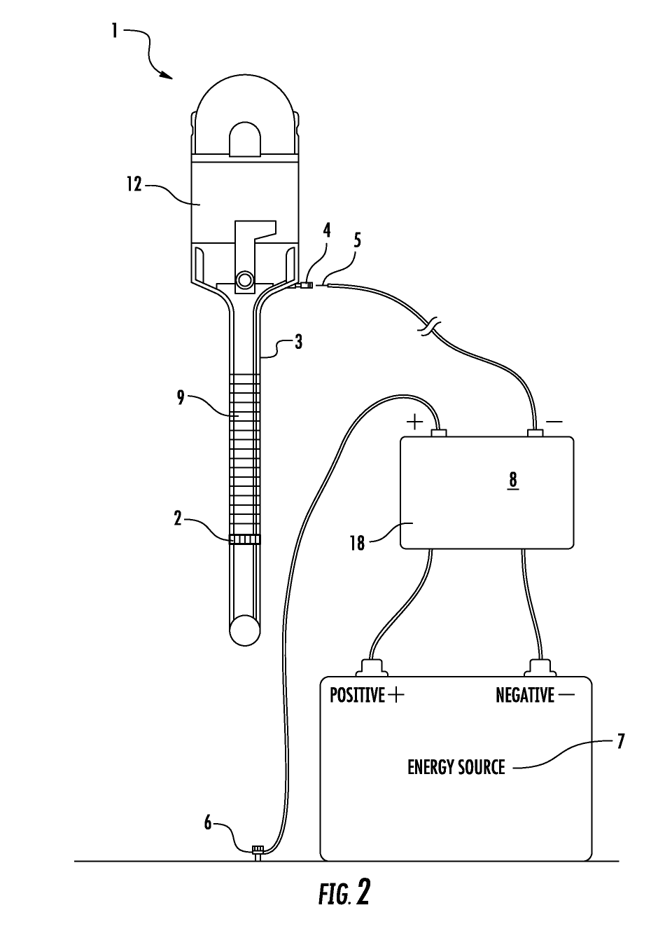

FIG. 2 shows that a DC energy source 7, such as a deep cycle, low voltage batteries supplies power to a DC boost converter 8. The boost converter 8 produces a much higher output voltage than it's original input voltage. For the purposes of this embodiment, the DC boost converter 8 acts as the high voltage source 18 for the device 1. The positive + pole of the high voltage source 18 is permanently connected to an isolated and unshared ground 6. The negative − pole of the high voltage source 18 is connected to the trocar 12 via an electrical plug 5 inserted into an electrical socket 4 affixed to or embedded in the trocar 12. Upon connection, there is continuity between the negative − pole of the high voltage source 18 and the carbon fiber electrode 2, via the electrical filament 3 running down the trocar shaft 9.

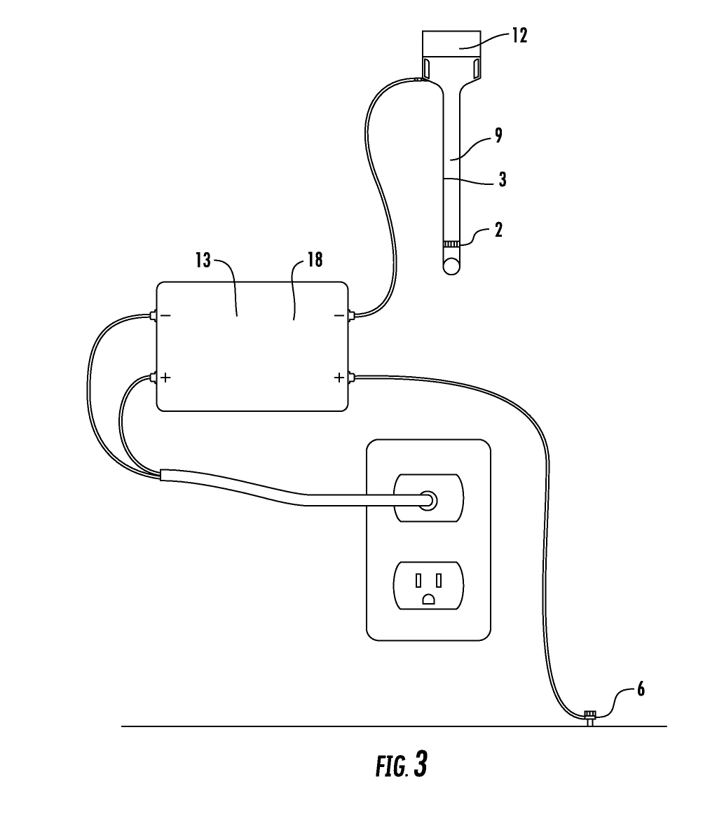

Turning to FIG. 3, it may be seen that the energy source 7 in this embodiment may be an alternating current (AC) wall socket 7 of varying voltage and phase. The energy source 7 supplies AC power to an AC/DC transformer 13, which transforms the current from AC to DC and outputs a higher DC voltage than it's AC voltage input. For the purpose of this embodiment the AC/DC transformer 13 is the high voltage source 18. The positive − pole of the high voltage source 18 is connected directly to an isolated and unshared ground 6. The negative − pole of the high voltage source is connected to the carbon fiber electrode 2 affixed to, or embedded in the trocar 12 in the exact same way as it is pictured in FIG. 2.

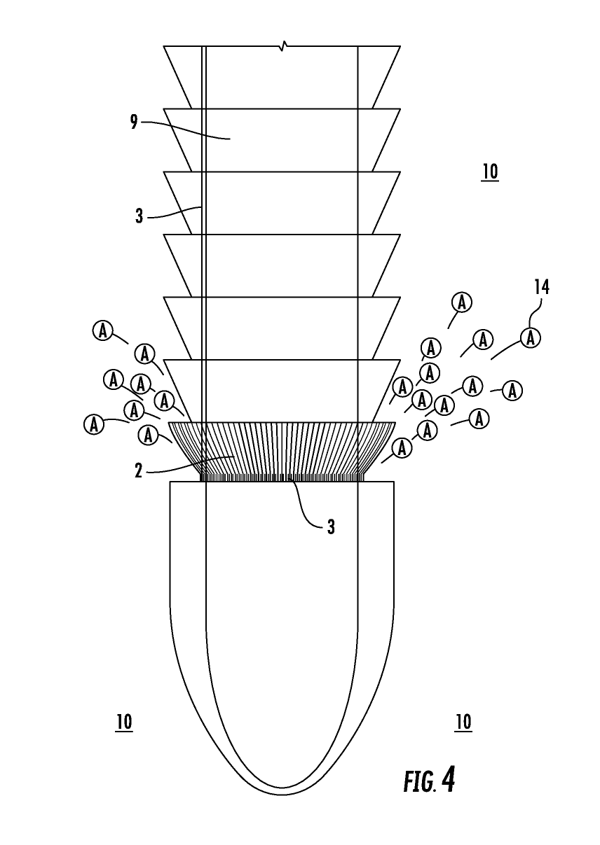

FIG. 4 illustrates the lower part of the trocar shaft 9, which penetrates the abdominal wall into the peritoneum. Illustrated is an electrical filament 3 with extremely low resistivity continuing the negative − pole of high voltage DC electricity down the trocar shaft 9 to the carbon fiber electrode 2. In the illustrated embodiment the electrical filament 3 and the electrode 2 encircle the trocar shaft 9 while making contact. When the device is operating, anions 14 stream from the terminal ends of the carbon fiber electrode 2 into the space of pneumoperitoneum 10.

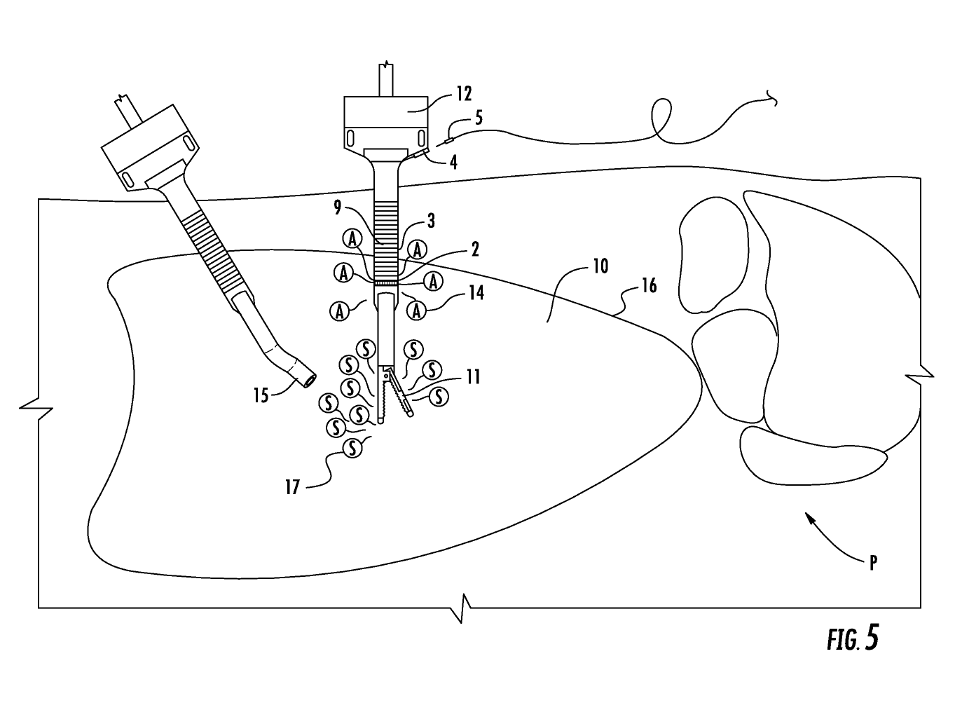

FIG. 5 shows the negative − pole of the high voltage source electrically connectable with the carbon fiber electrode 2 with the insertion of plug 5 into socket 4 bringing the carbon fiber electrode 2 into communication via the electrical filament 3, which runs down the trocar shaft 9. The trocar shaft 9 penetrates into the peritoneum 16 of the patient P and the trocar 12 acts as an access port for the electrosurgical and electrocautery devices 11 to pass into the pneumoperitoneum 10. The surgeon sees the surgical site through a camera called a laparoscope 15. As the electrosurgical and electrocautery devices burn and destroy tissue smoke S and suspended particles 17 begin to fill the pneumoperitoneum between the laparoscope 15 and the surgical site, obscuring the surgeon's view of target tissue as well as the electrosurgical and/or electrocautery device. When the present inventive apparatus is activated, anions 14 stream from the terminal points of the carbon fiber electrode 2 into the pneumoperitoneum 10. The anions (A) 14 react with the smoke (S) and suspended particles 17 causing the particles to come together and precipitate out of the suspending gas.

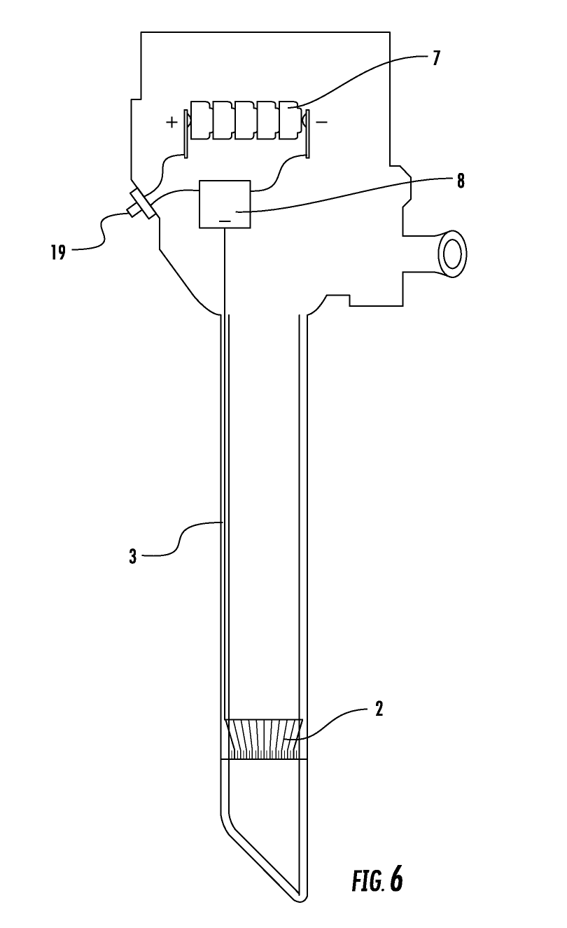

FIG. 6 illustrates the latest, smallest and most advanced working prototype. in this design, the entire apparatus is built within and on the trocar body. The battery or batteries 7 supply both positive and negative polarity low voltage DC electricity to a DC to DC Boost Converter 8. One of the legs is broken by an interruption switch 19, which is affixed to the outside of the trocar body and is used to interrupt and complete the circuit with the Boost Converter 8. The Boost Converter 8 produces high voltage DC electricity with negative polarity which runs down the trocar shaft 9 via the electrical filament 3 to one of the specified electrodes.

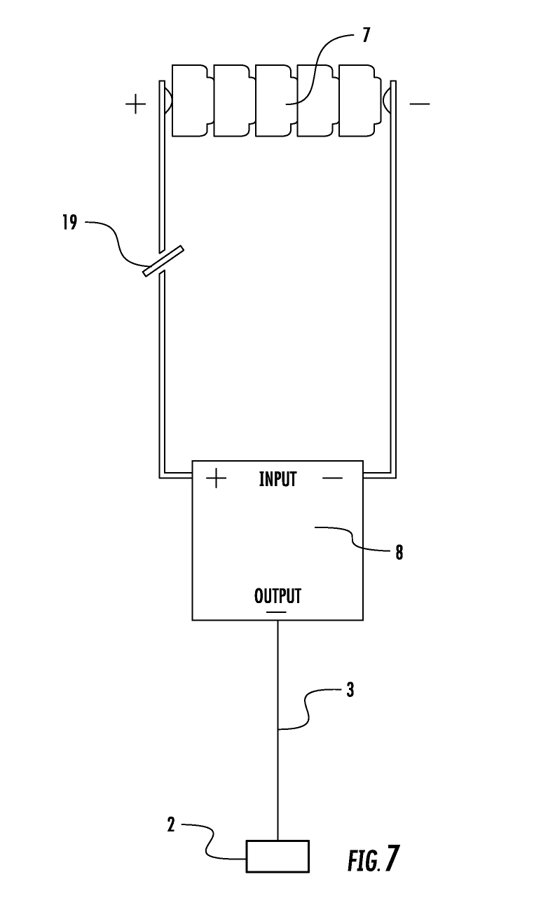

FIG. 7 is schematic of the apparatus illustrated in FIG. 6. It shows the battery or batteries 7, an interruption switch 19, a DC to DC Boost Converter 8, an electrical filament 3 carrying negative polarity high voltage DC electricity to the electrode 2.

The foregoing is considered as illustrative only of the principles of the invention. Further, since numerous modifications and changes will readily occur to those skilled in the art, it is not desired to limit the invention to the exact construction and operation shown and described.

Those skilled in the art will recognize other embodiments of the invention which may be drawn from the illustrations and the teachings herein. To the extent that such alternative embodiments are so drawn, it is intended that they shall fall within the ambit of protection of the claims appended hereto.

Having disclosed the invention in the foregoing specification and accompanying drawings in such a clear and concise manner, those skilled in the art will readily understand and easily practice the invention.

1. An apparatus for mitigation of smoke and particulate in minimally invasive surgery in an insufflated body cavity comprising:
a single carbon fiber or laser etched metal electrode in communication with a negative (neutral) pole of a source of high voltage direct current (DC) electricity; and
a positive pole of said source of DC electricity given a least resistive path to an isolated (unshared) ground wherein when said electrode is charged, anions (negative ions) stream from terminal ends of said carbon fibers into said insufflated body cavity and urge suspended smoke and particulate out of suspension.

2. A trocar comprising:
A battery or batteries operatively connected to an interruption switch, an optional voltage regulator, a DC to DC boost converter with a negative polarity output power supply, an electrical conduit of thin wire of highly conductive material; and an electrode of carbon fibers or laser etched metal with a multiplicity of terminal points from which anions may stream.

3. A method for mitigation of smoke and particulate in minimally invasive surgery in an insufflated body cavity comprising:
inserting into said body cavity a single electrode having a multiplicity of terminal points for anions to stream therefrom, said electrode in communication with a negative (neutral) pole of a source of high voltage direct current (DC) electricity;
electrically connecting a positive pole of said source of DC electricity to a least resistive path to an isolated (unshared) ground or a point of terminal insulation; and
charging said electrode to stream from terminal ends of the carbon fibers or etched metallic barbs of said electrode anions (negative ions) into said insufflated body cavity thereby urging suspended smoke and particulate out of suspension.
